(19) United States
(12) Patent Application Publication
(10) Pub. No.: US 2014/0187475 A1
(43) Pub. Date: Jul. 3, 2014

(54) Lantibiotic Compositions and Methods For Preventing or Treating Mastitis

(71) Applicant: Medela Holding AG, Baar (CH)

(72) Inventors: Thomas W. Hale, Amarillo, TX (US); Brian Warren, Schaumburg, IL (US); Brian Silver, Cary, IL (US)

(21) Appl. No.: 14/144,796

(22) Filed: Dec. 31, 2013

Related U.S. Application Data
(60) Provisional application No. 61/747,413, filed on Dec. 31, 2012.

(51) Int. Cl. A61K 38/16

(52) U.S. Cl. CPC A61K 38/164
USPC 514/2.9

(57) ABSTRACT

A composition suitable for application to the skin comprising at least one lantibiotic, an acidic external phase aqueous solution, at least one pharmaceutically acceptable internal phase carrier material, and a combination thereof. A method for preventing or treating mastitis, comprising administering to a human in need of such treatment a topical composition comprising a therapeutically-effective amount of at least one lantibiotic, an acidic aqueous solution, and at least one pharmaceutically acceptable carrier material.

### CROSS-REFERENCE TO RELATED APPLICATION

This application claims the benefit of the filing date of U.S. Provisional Patent Application Ser. No. 61/747,413, filed Dec. 31, 2012, which is hereby incorporated by reference in its entirety.

### FIELD OF THE INVENTION

The present invention relates generally to a composition to prevent or treat mastitis of the human breast and in particular a topically administered lantibiotic composition. It can also be used to treat and/or prevent nasal carriage in the mother and/or baby. The present invention is potentially useful in applications beyond breastfeeding, such as a pre-surgical treatment.

### BACKGROUND OF THE INVENTION

Mastitis may be defined as an infection of the tissue of the breast that occurs most frequently during the time of breastfeeding. Approximately 15-25% of breastfeeding mothers are afflicted with mastitis during early child rearing. This condition causes pain, swelling, redness, and increased temperature of the breast. It most often occurs when bacteria, often from the baby's mouth, enters a milk duct through a crack in the nipple, or through retrograde movement of milk from the nipple opening, back into the duct. Bacteria associated with causing mastitis in women is generally caused by gram-positive pathogens, including Staphylococcus aureus and Staphylococcus epidermidis. One example of such bacteria is Methicillin-resistant Staphylococcus aureus (MSRA), an antibiotic resistant form of Staphylococcus aureus.

The most widely used methods for the treatment of human mastitis include topical treatments of lanolin, warm compresses, herbal supplements, vitamin C for the treatment of symptoms, and most often, oral antibiotics. More recently, topical compositions of nisin prepared in water or 1-propanol have been administered to treat bovine or human mastitis. See Broadbent J R, Chou Y C, Gillies K, Kondo J K, Nisin inhibits several gram-positive, mastitis-causing pathogens, J. DAIRY SCI. December 1989; 72(12):3342-3345; Sears P M, Smith B S, Stewart W K, et al, Evaluation of a nisin-based germicidal formulation on teat skin of live cows, J. DAIRY SCI. November 1992; 75(11):3185-3190; Fernandez L, Delgado S, Herrero H, Maldonado A, Rodriguez J M, The bacteriocin nisin, an effective agent for the treatment of staphylococcal mastitis during lactation, J. HUM. LACT. August 2008; 24(3):311-316); Cotter, CURRENT PROTEIN AND PEPTIDE SCI. 2005; 6:61-75. Oral antibiotics, such as the cephalosporins, penicillins, and clindamycin, are commonly prescribed for the treatment of the infection.

However, these methods of treatment have many shortcomings. The topical compounds, herbal supplements and vitamins only treat the symptoms of mastitis, or in some cases, strengthen immunity against mastitis without addressing the infection. The nisin solutions used thus far are less bioactive at physiological and higher pH conditions at which they are applied, severely limiting their effective antimicrobial activity and decreasing their efficacy at treating mastitis. The major complication of using oral antibiotics includes the potential emergence of antibiotic-resistant strains.

Oral antibiotics, themselves, come with differing side effects for the mother and also sometimes the infant. Erythromycin, which is transferred into breastmilk in clinically significant amounts, provides an increased risk of pyloric stenosis in the infant in early postpartum. Other oral antibiotics may significantly increase the risk of superinfection with C. Difficile, a serious infection commonly found following the use of oral broad spectrum antibiotics.

Thus, there remains a need in this field for compounds and methods for effectively preventing mastitis by way of reducing the bacterial infection associated with the condition without adverse effects in the breastfeeding mother or her infant. In addition, the emollient properties of this cream also reduce the likelihood of trauma to the mother's nipple which is associated with an increase in the risk of developing mastitis.

### SUMMARY OF THE INVENTION

The present invention relates to a topical composition for the prevention and possible treatment of initial mastitis. In one aspect, the composition is suitable for application to the skin and comprises a therapeutically-effective amount of at least one lantibiotic, in an acidic aqueous solution, and at least one pharmaceutically acceptable topical carrier. Preferably, the pharmaceutically acceptable topical carrier is an odorless, tasteless, non-tacky ointment or cream for moisturizing the skin and providing soothing relief for sore or cracked nipples. In another embodiment, the topical carrier can be water, which is already part of the aqueous solution. The present invention also provides a method for preventing and treating mastitis by administering to a human in need of such treatment, a topical composition comprising a therapeutically-effective amount of at least one lantibiotic, in an acidic aqueous solution, and at least one pharmaceutically acceptable topical carrier. The composition is applied directly to sore, cracked, and inflamed nipples to kill the gram positive bacteria that are known to cause mastitis, in order to alleviate symptoms and reduce the healing time from the infection. In yet another embodiment, the composition is suitable for application to the skin and comprises a therapeutically-effective amount of at least one lantibiotic, in an acidic aqueous solution, and a skin care component such as an emollient and/or a moisturizer. This composition could be used for healing damaged or broken skin and/or reducing inflammation, cooling, or warming effects.

The topical nisin compositions of the present invention have improved solubility and maintained bioactivity in an acidic aqueous solution. Specific embodiments of the present invention will become evident from the following more detailed description of the invention and the claims.

### DETAILED DESCRIPTION OF EMBODIMENTS OF THE INVENTION

In a broad aspect, at least one lantibiotic is combined with an acidic aqueous solution in the external phase of the product and at least one pharmaceutically acceptable carrier material for use in the prevention and treatment of mastitis.

Lantibiotics are peptide-derived antimicrobial agents. Their name was introduced in 1988 as an abbreviation for lanthionine-containing antibiotic peptides.

Lanthionines consist of two alanine residues that are linked at their β-carbons by a thioether bridge. In lantibiotics, these lanthionines are imbedded within cyclic peptides. All lantibiotics that have been characterized with respect to the stereochemistry of the thioether linkage contain (2S,6R)-lanthionines (Lan), with many family members also containing (2S,3S,6R)-3-methyllanthionines. In addition, they typically (but not always) contain the unsaturated amino acids 2,3-dehydroalanine (Dha) and (Z)-2,3 dehydrobutyrine (Dhb). Cooper L E LBavdD, W., Biosynthesis and Mode of Action of Lantibiotic. In: Mander LaL, H-W ed. COMPREHENSIVE NATURAL PRODUCTS II: CHEMISTRY AND BIOLOGY. Amsterdam: Elsevier; 2010.

These compounds have antimicrobial activity. They can be used in therapies against antibiotic-resistant strains of bacteria. Lantibiotics include, but are not limited to, nisin (preferably nisin z), pep5, mersacidin, actagardine, streptin, cytolysin, epidermin, and any natural or bioengineered variants.

In one embodiment, the lantibiotic may be nisin z, which is a bacteriocin derived from Lactococcus lactis or chemically synthesized. Nisin z is a short, nontoxic peptide which exerts a bactericidal effect on many gram-positive organisms as well as some gram-negative bacteria, including Staphylococcus aureus, methicillin-resistant Staphylococcus aureus, most streptococcal species, and salmonella. The amount of nisin in the composition of this preferred embodiment may be less than or equal to about 250 parts per million (“ppm”).

The foregoing embodiment may also include an acidic aqueous solution. Acid solution systems serve to maintain the pH of compositions within a desired range. An acidic aqueous solution is composed of an acid with a base added (NaOH) to adjust the pH of the solution. Nisin is apparently most stable at low pH and has higher antimicrobial activity at a lower pH. The acidic aqueous solution of an embodiment of the present invention has an acidic pH to improve solubility and to maintain the biological activity of nisin in the composition.

In one embodiment, the acidic solution is an aqueous solution with citric acid. The pH of the aqueous solution may be adjusted with sodium hydroxide to about the pH of skin, or pH of 5.0. In this embodiment, the water content in the composition is high and the aqueous solution includes citric acid. This embodiment provides a proper environment for nisin activity and its transfer to the affected tissues. In a preferred embodiment, the pH of the water solution is about 5.0.

Most important in this embodiment is the principle that nisin must be placed in the external aqueous phase of this preparation. Nisin is an amphophilic molecule and when placed in an ointment preparation with little or no water, it has been found by our studies to be largely inactive as an antimicrobial. When placed in the external aqueous phase of an oil in water emulsion, it is highly active when placed on a skin or agar surface, because the nisin product directly dissipates on the skin or surface of the medium where it is placed. In an ointment preparation, it is retained by the lipid phase and fails to dissipate and kill bacteria.

The pharmaceutically acceptable internal carrier material may be an emollient to soften and smooth the skin. The pharmaceutically acceptable carrier material may comprise medium-chain triglycerides (“MCT”) oil, lanolin oil, olive oil, safflower oil, flaxseed oil, mineral oil, beeswax, cholesterol, petroleum jelly, Aquaphor®, or polyethylene glycol ointment NF as the internal phase. The carrier material can be formulated such that the composition of the present invention is a cream, spray or foam. The carrier may optionally contain small amounts of materials which enhance the texture and appearance of the composition as desired, for example, glycerin. These materials may also be emulsifier(s) such as cetyl alcohol and/or glyceryl stearate but not limited to these two examples.

In one embodiment, the pharmaceutically acceptable internal phase carrier material may comprise MCT oil (medium chain triglyceride oil). In another embodiment, the pharmaceutically acceptable carrier material may comprise lanolin. In a further embodiment, the pharmaceutically acceptable carrier material may comprise olive oil, cholesterol, and bees wax. In yet another embodiment the pharmaceutically acceptable carrier material may comprise flaxseed oil, safflower oil, coconut oil, cholesterol, and beeswax. In addition, alternative carrier materials are known in the art and may be used as long as they are formulated in the internal phase of the cream.

In another embodiment, the composition may also comprise at least one emulsifier, a thickener, and a preservative. The emulsifier(s) may comprise cetyl alcohol and/or glyceryl stearate. The thickener may comprise xanthan gum. The preservative may comprise potassium sorbate. In addition, alternative thickeners, emulsifiers, and preservatives are known in the art and may be used.

The composition of the present invention is capable of being administered to mammals in general and humans in particular. The invention is particularly useful when used multiple times per day before or after symptoms of the infection manifest or before or after nipples are sore or cracked or there has been a previous bout of mastitis. The emollient properties of this cream will provide lubrication to the skin of the nipple, thus reducing friction and trauma from repeated feedings during the day. Reducing the trauma to the nipple will reduce the risk of infection with oral microbes from the infant, or microbes from the skin of the mother's nipple.

In a further aspect, a method is provided for reducing inflammation, cooling or warming effects or for healing wounds or damaged or broken skin by administering to a human in need of such treatment a topical composition that includes a therapeutically-effective amount of at least one lantibiotic, an acidic aqueous solution, and at least one skin care component such as an emollient and/or a moisturizer.

In another aspect, a method is provided for preventing or treating mastitis by administering to a human in need of such treatment a topical composition that includes a therapeutically-effective amount of at least one lantibiotic, an acidic aqueous solution, and at least one pharmaceutically acceptable carrier material.

It should be understood that the foregoing disclosure emphasizes certain embodiments of the invention and that all modifications or alternatives equivalent thereto are within the spirit and scope of the invention as set forth in the appended claims.

### EXAMPLES

The Examples that follow are illustrative of specific embodiments of the invention, and various uses thereof. They are set forth for explanatory purposes only, and are not to be taken as limiting the invention.

In one non-limiting example, a composition is prepared by mixing a base, a preservative, an emollient, two emulsifiers, a thickener, and a preservative. The pH of the base is adjusted to about the pH of skin.

In another non-limiting example, a composition as shown in Table 1 below is prepared. The pH of the water solution is adjusted about the pH of skin.

TABLE 1
Ingredients

Water solution (water & citric acid & NaOH)
[CAS No. 5949-29-1]
[CAS No. 77-92-9]
Nisin, ≦250 ppm and >900 IU/mg
[CAS No. 1414-45-5]
MCT Oil
[CAS No. 73398-61-5]
Cetyl Alcohol
[CAS No. 36653-82-4]
Glyceryl Stearate
[CAS No. 31566-31-1]
Xanthan Gum
[CAS No. 11136-66-2]
Potassium Sorbate
[CAS No. 590-00-1]

While the invention has been described above according to its preferred embodiments, it can be modified within the spirit and scope of this disclosure. This application is therefore intended to cover any variations, uses, or adaptations of the invention using the general principles disclosed herein. Further, the application is intended to cover such departures from the present disclosure as come within the known or customary practice in the art to which this invention pertains and which fall within the limits of the following claims.

What is claimed is:

1. A composition suitable for application to the skin comprising of at least one lantibiotic, an acidic external phase aqueous solution, and at least one pharmaceutically acceptable internal phase carrier material.

2. The composition of claim 1, wherein the lantibiotic comprises nisin.

3. The composition of claim 2, wherein the amount of nisin in the composition is less than or equal to about 250 parts per million.

4. The composition of claim 2, wherein nisin has an activity of greater than about 900 IU/mg.

5. The composition of claim 1, wherein the acidic aqueous solution has a pH of about the pH of skin.

6. The composition of claim 1, wherein the external phase consists of an acidic solution of water, citric acid and sodium hydroxide.

7. The composition of claim 1, wherein the pharmaceutically acceptable internal phase carrier material comprises MCT oil.

8. The composition of claim 1, further comprising at least one emulsifier.

9. The composition of claim 1, further comprising a thickener.

10. The composition of claim 1, further comprising a preservative.

11. The composition of claim 1, further comprising comprising cetyl alcohol, glyceryl stearate, xanthan gum, and potassium sorbate.

12. A method for preventing or treating mastitis, comprising administering to a human in need of such treatment a topical composition comprising a therapeutically-effective amount of at least one lantibiotic, an acidic aqueous solution, and at least one pharmaceutically acceptable carrier material.

13. The method of claim 12, wherein the lantibiotic comprises nisin.

14. The method of claim 13, wherein the amount of nisin in the composition is less than or equal to about 250 parts per million.

15. The method of claim 13, wherein nisin has an activity of greater than about 900 IU/mg.

16. The method of claim 12, wherein the acidic aqueous solution has a pH of about the pH of skin.

17. The method of claim 12, wherein the acidic aqueous solution comprises water, citric acid and sodium hydroxide.

18. The method of claim 12, wherein the pharmaceutically acceptable carrier material comprises MCT oil.

19. The method of claim 12, further comprising at least one emulsifier.

20. A composition suitable for application to the skin comprising at least one lantibiotic, an acidic aqueous solution, and at least one skin care component such as an emollient and/or a moisturizer.
